(19) United States
(12) Patent Application Publication
(10) Pub. No.: US 2006/0148782 A1
(43) Pub. Date: Jul. 6, 2006

(54) Method and medicine for treating a mammal presenting urinary incontinence, urinary urgency, or both

(76) Inventor: James E. Lundeen, Cleveland Heights, OH (US)

Correspondence Address: RENNER OTTO BOISSELLE & SKLAR, LLP, 1621 EUCLID AVENUE, NINETEENTH FLOOR, CLEVELAND, OH, 44115, US

(21) Appl. No.: 10/970,174

(22) Filed: Oct. 21, 2004

Related U.S. Application Data
(60) Provisional application No. 60/518,715, filed on Nov. 10, 2003.
(60) Provisional application No. 60/518,718, filed on Nov. 10, 2003.
(60) Provisional application No. 60/518,719, filed on Nov. 10, 2003.

(51) Int. Cl. A61K 31/551; A61K 31/5415

(52) U.S. Cl. 514/220; 514/225.2

(57) ABSTRACT

A method and a medicine for treating a mammal, such as a person, cat, or dog having a urinary disorder that includes urinary incontinence and/or urinary urgency are provided. The method includes administering a dose of the medicine to the mammal. The medicine includes a tricyclic antidepressant and a stool softener.

### CROSS-REFERENCE TO RELATED APPLICATIONS

This application claims priority to U.S. Provisional Patent Application Ser. Nos. 60/518,715 filed on Nov. 10, 2003; 60/518,718 filed on Nov. 10, 2003; and 60/518,719 filed on Nov. 10, 2003, the disclosures of which are hereby incorporated by reference in their entirety.

### BACKGROUND

1. Field of Invention

The present invention relates to a method for reducing or eliminating urinary incontinence and/or urinary urgency. The present invention relates to a medicine for reducing or eliminating urinary incontinence and/or urinary urgency.

2. Discussion of Related Art

Urinary incontinence and/or urinary urgency are disorders that may afflict mammals. Mammals include, for example, people and domesticated animals such as cats and dogs. A review of a proper functioning system may illustrate the nature of the disorders. The kidneys make urine continuously by removing waste products and excess water from the bloodstream. The urine flows from the kidneys through the ureters to the urinary bladder. The bladder stores urine until full, and then signals the mammal that it is full. At this point, the mammal may go to the toilet to empty the bladder in a process called urination, micturition or voiding. Urination involves the simultaneous contraction of the bladder wall muscles and the relaxation of the muscles of the urinary sphincter. The urinary sphincter is a two part structure, that is, the bladder has an internal sphincter and an external sphincter that cooperate to control flow of fluid from the bladder to the urethra. The urine then leaves the body through the urethra.

Urinary urgency is the result of the signaling that the bladder is full or near full despite the actual level of fullness of the bladder or at a fill level that is undesirably lower than full. In addition, the urinary urgency may be the result of an overly strong signaling of the fullness of the bladder, suggesting a level of fullness greater than that actually present. Practically, a mammal suffering from urinary urgency feels an unwarranted urge to void the bladder either too often or too strongly, or both.

Urinary incontinence may include several types. The types include stress incontinence, urge incontinence, mixed incontinence, overflow incontinence, environmental incontinence, functional incontinence, post void incontinence, and nocturnal incontinence.

Stress incontinence is the loss of urine when pressure within the abdomen increases, and may be usually described as the involuntary loss of urine while coughing, sneezing, laughing, or lifting heavy objects. Poor bladder support by the pelvic muscles may result in the opening and descent of the urethra when abdominal pressure is increased. Types I, IIa, and IIb incontinence are progressive forms of this urethral “hypermobility.” Childbearing predisposes some women to this type of incontinence. Intrinsic sphincter deficiency (Type III incontinence) is another cause of stress incontinence due to a nonfunctional proximal urethra that remains open at all times. It may be caused by prior surgery or by changes in the urethral tissues from aging.

Urge incontinence may involve the sudden sensation of a need to urinate with inability to get to a toilet before involuntary leakage occurs. At times, urine may leak without any warning. This condition may be due to an overactive bladder that suddenly contracts without the mammal's desire for it to do so. This condition may be the result of damage to nerve passages that connect the bladder to the brain. Causes of urge incontinence may include injury, bladder infections that irritate the bladder lining, and neurologic problems that affect bladder function, such as a stroke, dementia, a mechanically unstable spine, and multiple sclerosis. In some mammals, the cause remains unclear. Mixed incontinence may be a combination of both stress and urge incontinence, causing symptoms of both.

Overflow incontinence may occur when the bladder is so full that it leaks urine, or “overflows.” This can happen when a blocked urethra prevents the bladder from emptying normally, such as in enlargement of the prostate. Overflow incontinence also may happen if the bladder is too weak to contract and to force the urine out, such as in diabetes or certain neurologic disorders.

Environmental incontinence occurs when a mammal is unable to get to a toilet on time due to extrinsic factors. The urinary system may work well, but circumstances prevent normal toilet usage.

People with functional incontinence may have problems thinking, moving, or communicating that prevent them from reaching a toilet. A person with Alzheimer's disease, for example, may not think well enough to plan a timely trip to a restroom. A person in a wheelchair may be blocked from getting to a toilet in time. Conditions such as these may be associated with age and account for some of the incontinence of elderly women in nursing homes.

Post void incontinence may include the inability to control continence of the post void residual (PVR) amount of urine in the bladder. That is, after voiding the bladder, a residual amount of urine may be still contained in the bladder, which will leak from the bladder involuntarily shortly after the voiding. This secondary release of urine may occur within seconds or minutes of the initial voiding.

Nocturnal incontinence, enuresis, or nocturia may include the inability of a mammal to hold urine in the bladder during a sleep cycle. This disorder may be common among older children.

The least invasive methods of bladder control may be therapeutic. Therapeutic methods may involve making changes in behavior patterns and performing bladder muscle-strengthening exercises. When incontinence does not respond to therapeutic bladder control methods, the next step may be medical—using medicinal compositions or medicines, and hormones. Surgical methods may be used only after all other bladder control methods fail or if the cause of incontinence can only be treated surgically.

With reference to medicines used to treat urinary incontinence, it may be desirable to have improved medicines. Conventional medicines may include drugs from the alpha-adrenergic agonist family. These may drugs help the urinary sphincter maintain tone and contract with greater strength, thereby preventing urinary leakage.

Other drugs may combat urge incontinence, such as oxybutynin, calcium channel blockers, anti-cholinergic medications and tricyclic antidepressants. These drugs relax the involuntary contractions thought responsible for urge incontinence. Treatment effectiveness may be limited by side effects, and in particular, tricyclic antidepressants may cause undesirable constipation. The development of controlled-release medications was an attempt to limit side effects while maintaining effectiveness. Older children who suffer from bedwetting may be prescribed tricyclic antidepressants, but must be monitored closely for side effects. Desmopresin may be prescribed for urge incontinence, as it may reduce nighttime urine production. Involuntary contractions may be caused by a condition known as detrusor hyperreflexia. Treatment for detrusor hyperreflexia includes administration of intravesical resiniferatoxin, which is a capsaicin analog.

Postmenopausal women suffering from bladder control problems may undergo estrogen therapy. The hormone estrogen plays a role in maintaining the strength and tone of the pelvic floor muscles in women. Menopause, and the accompanying reduction in the body's estrogen levels, can lead to weakened pelvic muscles and stress incontinence. Estrogen therapy may help to reverse this trend.

Damage or injury to bladder sphincters may be a result of surgery, child-birth, or trauma. Underperforming bladder sphincters may be a result of malnutrition, diabetes, cancer and or damage to the spinal cord.

Collagen injections are another option available to both men and women with bladder control problems. Collagen is injected into the walls of the urethra close to the urinary sphincter. This builds up the area around the sphincter, compressing the sphincter and making urine retention easier.

In spite of conventional treatments, compositions and methods used to reduce or eliminate urinary incontinence and/or urinary urgency no suitable long term, efficacious treatment or preventative has been identified. It would be desirable to have a medicinal composition or medicine having improved properties for the treatment of urinary incontinence and/or urinary urgency. It would be desirable to have a method of treatment for of urinary incontinence and/or urinary urgency.

### SUMMARY

An aspect of the present invention relates to a method for treating a mammal having urinary incontinence and/or urinary urgency. Another aspect of the present invention relates to a medicine for treating a mammal having urinary incontinence and/or urinary urgency.

Another aspect of the invention relates to a method, which includes administering a dose of the medicine to the mammal, the medicine includes a tricyclic antidepressant and a stool softener.

Yet another aspect of the invention relates to a process that includes interacting with muscarinic receptors in the mammal to reduce or eliminate urinary incontinence and/or urinary urgency, and emulsifying oil and water into fecal matter using the surfactant to soften the stool of the mammal, lubricating the fecal matter to facilitate passage of the stool, or both emulsifying and lubricating the fecal matter to both soften the stool and facilitate passage of the stool.

### BRIEF DESCRIPTION OF THE DRAWINGS

FIG. 1 is a schematic diagram showing a packaging configuration of a medicine comprising an embodiment in accordance with the invention; and

FIG. 2 is a schematic block diagram showing a method in accordance with the invention.

### DESCRIPTION OF PREFERRED EMBODIMENTS

The present invention relates to a method of treating urinary incontinence and/or urinary urgency in a mammal. In one embodiment of the invention, the mammal may be a person, and in another embodiment the mammal may be a domesticated pet, such as a dog or a cat. The present invention also relates to a medicinal composition or medicine for use treating urinary incontinence and/or urinary urgency in a mammal.

A method according to embodiments of the invention includes the administration of a dose or a series of doses of the medicine to a mammal suffering from or presenting symptoms associated urinary incontinence and/or urinary urgency. The medicine is described below, as is dosage information. The urinary incontinence may be stress incontinence, urge incontinence, mixed incontinence, functional incontinence, and nocturnal incontinence. For convenience, unless otherwise indicated specifically or by context, urinary incontinence and urinary urgency may be collectively referred to as “urinary incontinence”.

The medicine includes a tricyclic antidepressant and one or both of a stool softener and a fecal lubricant. Tricyclic antidepressants may be used alone or in combination and may include amitriptyline, clomipramine, desipramine, imipramine, doxepin, and nortriptyline, and derivatives and pharmaceutically acceptable salts thereof. Unless otherwise specified or indicated by context, “stool softener” will herein collectively include both stool softener and fecal lubricant for ease of referral.

In one embodiment, the tricyclic antidepressant includes imipramine (5-[3-(dimethylamino)propyl]-10,11-dihydro-5H-dibenzazepine), which is shown structurally below, or an active metabolite thereof—such as desmethylimipramine.

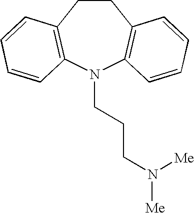

In another embodiment, the tricyclic antidepressant includes imipramine HCl. Imipramine hydrochloride is available for commercial sale as TOFRANIL from Mallinckrodt Inc. (St. Louis, Mo.). As used throughout, reference to dosage of imipramine generally will be to an equivalent amount of imipramine HCl specifically. Also, unless specified, dosage values are in units of milligrams. For anti-depression treatment, the manufacture supplies TOFRANIL in 10, 25, 50 and 75 mg tablets or capsules.

In yet another embodiment, the tricyclic antidepressant includes imipramine pamoate (5-[3-(dimethylamino)]propyl-10,11-dihydro-5H-dibenzazepine 4,4-methlyenebis-(3-hydroxy-2-napthoate) (2:1 ratio of pamoate to imipramine). imipramine pamoate is commercially available as TOFRANIL-PM from Mallinckrodt Inc. (St. Louis, Mo.).

The total daily dosage of imipramine in a medicine according to the present invention may be in a range of from about 5 mg/day to about 200 mg/day. In one embodiment, the amount may be in a range of from about 5 mg/day to about 25 mg/day, from about 25 mg/day to about 50 mg/day, from about 50 mg/day to about 75 mg/day, from about 75 mg/day to about 125 mg/day, or from about 125 mg/day to about 150 mg/day. Here and elsewhere, range limitations may be combined.

Alternatively, in one embodiment the total daily dosage may be based on patient weight. According to an embodiment of the present invention a total daily dosage of imipramine in a medicine may be in a range of from about 0.1 milligram/kilogram body weight/day (mg/kg/day) to about 2.5 mg/kg/day, 0.2 mg/kg/day to 1.2 mg/kg/day, about 0.5 mg/kg/day to about 2.0 mg/kg/day, about 0.5 mg/kg/day to about 0.75 mg/kg/day, about 0.75 mg/kg/day to about 1.25 mg/kg/day, or about 1.25 mg/kg/day to about 2.0 mg/kg/day.

Because children may have a relatively higher glomerular filtration rate (GFR) the dosage may need to be adjusted upward to accommodate such, rather than downward as seen in anti-depression treatment. In one embodiment, a child dosage may be up four times greater than the adult dosages, or may be up to about 400 mg/day. For elderly, infirm, or smaller than average-sized mammals a total daily dosage amount may be adjusted downward, for example, in a range of from about 5 mg to about 25 mg.

Relatively decreased dosages of, for example, imipramine pamoate having a comparable effect as higher dosages may be achieved by concurrently ingesting metabolism inhibiting compositions, such as methylphenidate HCl (which is commercially available from Ciba-Geigy Corporation (Basel, Switzerland), a division of Novartis Pharmaceuticals Corporation, as RITALIN and RITALIN SR).

The stool softener used herein is distinguished from laxatives. Laxatives include bulk, osmotic and stimulant-type. Bulk laxatives include soluble and insoluble fiber. Soluble fiber can include psyllium husks and is commercially available as METAMUCIL from Procter & Gamble Inc. (Cincinnati, Ohio). Insoluble fiber can include wheat bran. Osmotic laxatives are not absorbed and function by pulling water into the colon via osmotic action (e.g., magnesium hydroxide, such as PHILLIP'S MILK OF MAGNESIA, which is commercially available from Bayer Corporation (Pittsburgh, Pa.)). Stimulant laxatives interfere with absorption of water from the colon lumen and motility of fecal material therethrough.

By way of contrast, a stool softener acts to emulsify water and/or oil into fecal matter and thus may soften the consistency. A fecal lubricant acts by lubricating the fecal matter and allowing it to pass though the colon with a reduced amount of friction. Suitable stool softeners include surfactants, such as anionic surfactants, nonionic surfactants, cationic surfactants, and ampholytic surfactants. Particularly suited surfactants include anionic surfactants. In one embodiment, the stool softener includes bis (2-ethylhexyl) sulfosuccinate sodium salt (“docusate sodium”), which is commercially available from Purdue Phama L.P. (Stamford, Conn.) as COLACE. Other suitable metal salts of sulfosuccinate are also useful, and the metal may be potassium, calcium and the like. PERICOLACE (which is a tradename for docusate plus casanthrol), sodium dodecylsulfate (SDS), sodium cholate, sodium deoxycholate (DOC), N-lauroylsarcosine sodium salt, lauryldimethylamine-oxide (LDAO), and cetyltrimethyl ammoniumbromide (CTAB) may be used in embodiments according to the invention.

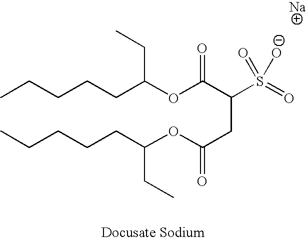

The fecal lubricant may include, for example, commercially available mineral oil or liquid paraffin. The stool softener and fecal lubricant may be used alone and in combination with each other. In combination, the stool softener can emulsify the fecal lubricant into the stool.

In one embodiment according to the invention, the stool softener may be used in the manufacturer's recommended dosage levels of less than 200 mg/day, the manufacturer's recommended dosage level for COLACE is about 50-200 mg/day in divided doses. In another embodiment, the dosage of stool softener may be in a range of from about 200 mg/day to about 400 mg/day, and may be used in an amount of about 300 mg/day, or up to about 400 mg/day.

Alternatively, the amount of the stool softener may be determined with reference to body weight. In one embodiment, the total daily dosage may be in a range of from about 1 mg/kg/day to about 4 mg/kg/day. In one embodiment, the total daily dosage may be in a range of from about 1.0 mg/kg/day to about 2.0 mg/kg/day, from about 2.0 mg/kg/day to about 3.0 mg/kg/day, or from about 3.0 mg/kg/day to about 4.0 mg/kg/day.

The dosage amount of tricyclic antidepressant to stool softener may be expressed as a ratio or a proportion. In one embodiment, the ratio of tricyclic antidepressant to stool softener is in a range of from about 1:80 to about 3:1, from about 1:12 to about 1:6, from about 1:4 to about 1:3, from about 1:2 to about 1:1, or from about 2:1 to about 3:1. In one embodiment, the ratio may be preselected based on weight, symptom severity, symptom type, symptom frequency, dietary considerations, type of tricyclic antidepressant and stool softener, dose regimen, administration method, environmental considerations, other or additional medications, and the like. In one embodiment, the ratio may be selected based on individual responsiveness, dietary considerations, environmental considerations, side effects, aggravating conditions such as stress level, other or additional medications, and the like.

The frequency of stool softener dosages may be determined on an individual basis. However, in one embodiment the daily dosage is 300 mg/day taken in three 100 mg doses spaced over the course of the day, optionally with a meal. As noted above the stool softener may be taken concomitant with the tricyclic antidepressant or may be taken at a different time relative to the tricyclic antidepressant. The daily dose(s) of tricyclic antidepressant may be taken as a single dose or as multiple portions over the course of the day. For example, a 75 mg/day dose may be taken as 25 mg three times a day, optionally with meals. The tricyclic antidepressant may be taken concomitant or concurrent with the stool softener or may be taken at a time different than the stool softener.

The tricyclic antidepressant portion and the stool softener portion of each dose that forms the total daily dose may be packaged in fractional dose amounts. The fractional doses may be combined and taken substantially at the same time to form a full dose, one or more full doses may be combined to form a total daily dose. For example, if the medicine is in the form of admixed pills that each contain 50 mg of stool softener and 5 mg of tricyclic antidepressant, and the total daily dose is 300 mg of stool softener and 30 mg of tricyclic antidepressant, to be taken in three doses over the course of a day. Each of three doses can include two of the fractional doses (10 mg of tricyclic antidepressant and 100 mg of stool softener). The number of fractional doses taken may be varied over the course of a day, or may be varied over the course of the treatment. The number of fractional doses taken (and thus the amount of the total daily dose) can be selected to correspond to preselected factors. Such factors may include, for example, seasonal changes (e.g., dehydration being more prevalent in summer months may result in a reduced amount of urine produced), aging, the natural course of the urinary disorder, stress inducing situations, and others that may affect the occurrence or severity of symptoms of the urinary disorder.

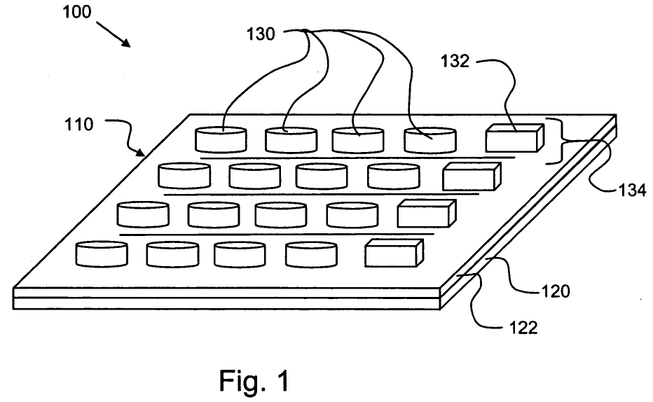

With reference to FIG. 1, a packaged treatment regimen 100 showing an embodiment according to the invention includes a blister pack 110. The blister pack 110 has a base layer 120 secured to a bottom surface of a top layer 122. The top layer 122 defines storage blisters, and the base layer 120 can operate to seal the blisters to releasably contain doses of the medicine, or portions of the medicine. The blisters in the illustrated embodiment define differing shapes merely for the purpose of ease of differentiation. In the embodiment shown, stool softener may be housed in the blisters labeled 130, and the tricyclic antidepressant is housed in the blister labeled 132. A row or strip 134 may equal a total daily dose of the medicine. Because in the illustrated embodiment, the total daily dose includes four portions of stool softener (at, for example, 75 mg each) and one portion of tricyclic antidepressant (at, for example, 25 mg), there are correspondingly four blisters 130 for housing the stool softener and one blister 132 for housing the tricyclic antidepressant. Thus, the stool softener may be taken four times a day for 300 mg/day total daily dose, and the tricyclic antidepressant may be taken once a day for 25 mg/day total daily dose. Furthermore, the tricyclic antidepressant may be taken with one of the stool softener doses or at another time, as desired. The strip 134 is one of four shown on the blister package 110, which is a four day supply of medicine. The blister package 110 may have instructions printed thereon that indicate what the dosage regimen may be, and, optionally and/or additionally, directions for varying portion dosage with reference to symptomology or exacerbating conditions.

In other embodiments, the tricyclic antidepressant and stool softener portions may include dosages having differing amounts for different total daily dosages, may have differing numbers of doses for the same or different total daily dosages, and may have doses that include both the tricyclic antidepressant and the stool softener in a single form (such as a pill containing both the tricyclic antidepressant and the stool softener). Further, other embodiments according to the invention may have the tricyclic antidepressant and/or the stool softener in a form other than pill, gel cap, and the like, and may not be amenable to blister packaging. Suitable packaging may then be selected based on the form of the tricyclic antidepressant and the stool softener, and whether the tricyclic antidepressant and the stool softener are admixed or physically separate.

With reference to form and the packaging, at least a portion of the medicine is in the form of a pill, capsule, gelcap, a coated or chewable tablet, a chewable gum, an ingestible liquid admixture, transdermal patch, an inhalable powder or mist, an enema or suppository, an intravenous solution or an intramuscular injectable liquid. Administering the dose includes selecting an entry method into the mammal based on the form of the medicine. For example, if imipramine is provided as a gelcap, and sodium docusate is provided as an ingestible liquid, the imipramine may be swallowed and the sodium docusate may be imbibed either at about the same time or at different times during a day. Imipramine and sodium docusate may be combined in a single capsule, in which case the capsule may be taken once a day or as part of a series of capsules taken throughout a day depending on the dosage amount in each capsule. For pills, capsules, gelcaps, tablets, and the like, suitable packaging includes multi-dose packages, such as blister packs. The blister packs may contain dosages of the medicine according to the present invention.

With reference to forms of the medicine other than those discussed above, the ingestible liquid admixture may be administered in pre-measured amounts. The transdermal patch, the chewable gum, the intravenous solution, or the intramuscular injectable liquid, and the oral and/or nasal inhaler (for the inhalable powder or mist) may be used to deliver the tricyclic antidepressant, while the stool softener may be administered via a different method. The enema or suppository may contain the stool softener and may be administered in a conventional manner. For orally administrable embodiments in which at least one component or portion of the medicine is taken orally, masking agents may be used. For example, edible carriers, such as food, may be used to enhance palatability of the medicine or medicine component. In one embodiment, the food is selected to have a pharmacological effect. For example, prune juice has a known tendency to increase bowel movement frequency, and this tendency may be factored into the dosage amounts for the medicine or medicine components.

In one embodiment, the medicine may contain additional material either admixed or separate from the tricyclic antidepressant, the stool softener, or both. For example, the medicine may contain a skeletal muscle relaxant, a narcotic, or a proton pump inhibitor, and may further include a suitable pharmaceutical excipient, diluent, or carrier selected with regard to the intended route of administration and standard pharmaceutical practice. Suitable skeletal muscle relaxants include cyclobenzaprine Hydrochloride, which is classified as a tricyclic antidepressant and is commercially available from McNeil Corporation (Fort Washington, Pa.) as FLEXERIL. cyclobenzaprine hydrochloride may be combined in the medicine according to the invention. A useful dose of cyclobenzaprine hydrochloride may be 10 milligrams 4 times a day. A dosage upper limit may be about 40 milligrams a day.

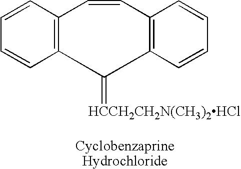

Suitable narcotics include opioid agonists include PERCOCET (oxycondone plus acetaminophen), which is commercially available from Endo Laboratories, Inc. (Chadds Ford, Pa.). Suitable proton pump inhibitors include omeprazole or 5-methoxy-2-[[(4-methoxy-3,5-dimethyl-2-pyridinyl)methyl]sulfinyl]-1H-benzimidazole, which is commercially available from AstraZeneca LP (Wilmington, Del.) as PRILOSEC, and lansoprazole, which is commercially available from TAP Pharmaceutical Products Inc. (Lake Forrest, Ill.) as PREVACID.

In one embodiment, the medicine further includes a beta-blocker, such as atenolol, which is commercially available from Medley Pharmaceuticals, Ltd (Maharashtra, India) as TENORMIN. Atenolol is a synthetic, beta1-selective (cardioselective) adrenoreceptor blocking agent or “beta-blocker”, that may be chemically described as benzeneacetamide, 4-[2′-hydroxy-3′-[(1-methylethyl)amino]propoxy]benzeneacetamide. Atenolol may block the action of the sympathetic nervous system. Because the sympathetic nervous system controls or influences the pace of the heart beat, blocking the action of these nerves can reduce the heart rate. Atenolol may reduce the force of heart muscle contraction, lower blood pressure, and may affect symptoms associated with urinary incontinence and/or urinary urgency, such as bowel frequency. Where tachycardia may be caused, for example, as a result of the action of the tricyclic antidepressant, a beta-blocker such as atenolol may be used to maintain the heart rate in a desired range.

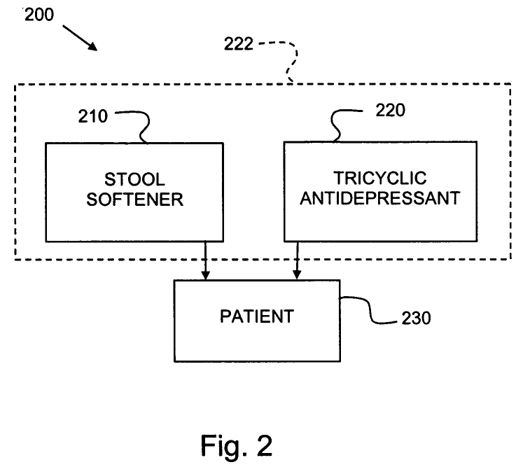

With reference to FIG. 2, a method according to the present invention is shown as a block diagram 200. A stool softener 210 and a tricyclic antidepressant 220 comprise a medicine 222. The stool softener 210 and the tricyclic antidepressant 220 are administered to a patient 230 suffering from a urinary disorder in accordance with the invention.

During use, the tricyclic antidepressant may improve the performance of the urinary internal sphincter. In one embodiment, the resting pressure of the internal urinary sphincter is increased in the presence of a sufficient blood level of imipramine. Increasing such performance may compensate for an underperforming external sphincter, weak pelvic musculature, overactive bladder musculature, and relaxation of the urinary sphincters during deep sleep. But, other sphincters may be affected also, such as anal sphincters as well as the gastrointestinal tract, thus causing constipation or obstipation. To alleviate this problem, the stool softener is present. The stool softener may modify the consistency and hardness of the stool to allow for easier passage and voiding of fecal matter.

### EXAMPLES

Embodiments according to the invention are illustrated in the following examples. In particular, the treatment of urinary incontinence and/or urinary urgency by methods and with medicines according to the present invention is shown.

### Example 1

A Caucasian female patient, 33 years old, presents with workplace injuries of sprain cervical spine and sprain lumbar spine. The spinal injuries relate to neck and back pain, with spasms, fecal urgency, urinary incontinence and/or urinary urgency, urinary urgency, and urinary incontinence. On presentment, protective pads are worn to absorb urine and feces.

The patient is treated with a daily dose of medicine, which includes 75 mg of imipramine pamoate and a stool softener. After several days of daily treatment via oral administration, the patient notes control of urinary incontinence and/or urinary urgency. The patient is able to stop using protective absorbent pads during treatment. The patient reports no adverse side affects, and in particular denies dry mouth and dry eyes.

### Example 2

A Caucasian female patient, 28 years old, presents with sprain lumbar spine. The spinal injury relates to back pain, with spasms, stress urinary incontinence and urinary urgency. For example, a sneeze may result in bladder incontinence. On presentment, protective pads are worn to absorb urine and feces.

The patient is treated with a daily dose of medicine, which includes 75 mg of imipramine pamoate and a stool softener. After several days of daily treatment via oral administration, the patient notes control of urinary incontinence and/or urinary urgency. The patient stops using protective absorbent pads. The patient tolerates a dry mouth.

### Example 3

A Caucasian female patient, 43 years old, presents with sprain lumbar spine. The spinal injury relates to back pain, with spasms, urgency and incontinence of the bladder and bowel.

The patient is treated with a daily dose of medicine, which includes 75 mg of imipramine pamoate. After several days of daily treatment via oral administration, the patient notes full control of bladder functions. The patient develops constipation and is prescribed docusate sodium (COLACE) in conjunction with the imipramine pamoate. The constipation is relieved by the docusate sodium.

The patient stops taking the daily dosages. After about three days without treatment, the bladder urgency and incontinence recur.

### Example 4

A Caucasian male patient, 45 years old, presents with sprain of sacrum, lumbar disc displacement, sprain lumbosacral, recurrent depression (psych-severe), and gastritis. The complaints include pain, spasms, depression, upset stomach, urinary incontinence and/or urinary urgency, and fecal and urinary incontinence (awake and sleeping).

The patient is treated with a total daily dose of medicine, which includes 75 mg of imipramine hydrochloride (25 mg/3 times daily) and a stool softener. After several days of daily treatment via oral administration, the patient notes partial to full control of bladder functions (awake and sleeping) and a reduction or elimination of urinary incontinence and/or urinary urgency symptoms. The patient tolerates a dry mouth. The patient switches to 75 mg/day (1 dosage/day) of imipramine pamoate, and a stool softener, with continued full control of bladder functions (awake and sleeping) and a reduction or elimination of urinary incontinence and/or urinary urgency symptoms.

### Example 5

A male patient presents with a nerve injury to the spine. The complaints include urinary incontinence and/or urinary urgency (awake and sleeping).

The patient is treated with a total daily dose of medicine, which includes 50 mg of tricyclic antidepressant (imipramine hydrochloride) and 300 mg of stool softener (docusate sodium). After several days of daily treatment via oral administration, the patient notes full control of bladder functions (awake and sleeping). That is, a reduction or elimination of urinary incontinence and/or urinary urgency symptoms.

### Example 6

A female patient presents with a pelvic nerve injury. The complaints include chronic urinary incontinence and/or urinary urgency.

The patient is treated with a total daily dose of medicine, which includes 50 mg of imipramine hydrochloride and 350 mg of stool softener (docusate sodium), ingested separately. After several days of daily treatment via oral administration, the patient notes partial to full control of bladder functions (awake and sleeping). That is, a reduction or elimination of urinary incontinence and/or urinary urgency.

### Example 7

A male patient presents with a compromised vascular supply to the bladder. The complaints include chronic, intermittent urinary incontinence and urinary urgency.

The patient is treated with a total daily dose of medicine, which includes an admixture of 25 mg of imipramine hydrochloride and 275 mg of stool softener (docusate sodium). After several days of daily treatment via oral administration, the patient notes partial to full control of bladder functions (awake and sleeping). That is, a reduction or elimination of urinary incontinence and urinary urgency symptoms. A selective beta-blocker is administered in response to tachycardia on an as-needed basis.

### Example 8

A group of twenty mammals, including female and male cats and dogs, present with urinary incontinence.

Each of the group is treated with a total daily dose of medicine, which includes an admixture of 5 mg of imipramine hydrochloride and 100 mg of stool softener, or in some instances the amounts of imipramine and stool softener are determined based on weight. After several days of daily treatment via oral administration it is noted that there is a reduction or elimination of urinary incontinence for a significant portion of the members of the group. That is, it appears that partial to full control of bladder functions (awake and sleeping) is achieved. That is, a reduction or elimination of urinary incontinence is apparent.

The processes and embodiments described herein are examples of compositions, systems and methods having elements corresponding to the elements of the invention recited in the claims. This written description may enable those skilled in the art to make and use embodiments having alternative elements that likewise correspond to the elements of the invention recited in the claims. The intended scope of the invention thus includes other compositions, systems and methods that do not differ from the literal language of the claims, and further includes other compositions, systems and methods that include equivalents of, or have insubstantial differences from, the literal language of the claims.

What is claimed is:

1. A method of treating a mammal having urinary incontinence, the method comprising:
administering a dose of a medicine to the mammal having urinary incontinence, the medicine comprising
a tricyclic antidepressant, and
a stool softener.

2. The method as defined in claim 1, wherein the urinary incontinence comprises stress incontinence, urge incontinence, mixed incontinence, post void incontinence, functional incontinence, nocturnal incontinence, or a combination of two or more thereof.

3. The method as defined in claim 2, wherein the urinary incontinence comprises stress incontinence or functional incontinence.

4. The method as defined in claim 1, wherein the urinary incontinence is associated with urinary urgency.

5. The method as defined in claim 1, wherein the mammal is human.

6. The method as defined in claim 1, wherein the mammal is a cat or a dog.

7. The method as defined in claim 1, wherein the tricyclic antidepressant is present in an amount in a range of about 5 milligrams per day to about 200 milligrams per day.

8. The method as defined in claim 7, wherein the tricyclic antidepressant is present in an amount in a range of about 25 milligrams per day to about 75 milligrams per day.

9. The method as defined in claim 1, wherein the stool softener is present in an amount in a range of about 100 milligrams per day to about 400 milligrams per day.

10. The method as defined in claim 9, wherein the stool softener is present in an amount in a range of about 200 milligrams per day to about 300 milligrams per day.

11. The method as defined in claim 1, wherein the tricyclic antidepressant comprises imipramine hydrochloride, imipramine pamoate, a pharmacologically acceptable salt of imipramine, or combinations of two or more thereof.

12. The method as defined in claim 1, wherein the mammal is a non-elderly adult human.

13. The method as defined in claim 12, wherein the human is not clinically depressed.

14. The method as defined in claim 1, wherein the stool softener comprises an anionic surfactant.

15. The method as defined in claim 14, wherein the surfactant comprises docusate sodium.

16. The method as defined in claim 15, wherein the stool softener comprises a fecal lubricant.

17. The method as defined in claim 1, wherein the urinary disorder is a chronic condition, and the administering is performed over an extended period of time corresponding to a treatment of the chronic condition.

18. The method as defined in claim 1, wherein the tricyclic antidepressant and the stool softener are adjacent to each other, spaced from each other, or admixed with each other while in the medicine.

19. The method as defined in claim 1, further comprising forming at least a portion of the medicine as a pill, capsule, gelcap, an ingestible liquid admixture, transdermal patch, an inhalable powder or mist, an enema or suppository, a coated or chewable tablet, a chewable gum, an intravenous solution, or an intramuscular injectable liquid, and administering comprises selecting an entry method into the mammal based on the form of the medicine.

20. The method as defined in claim 1, further comprising administering the tricyclic antidepressant and the stool softener substantially simultaneously or sequentially relative to each other.

21. The method as defined in claim 1, further comprising varying amounts of the tricyclic antidepressant and the stool softener over a course of treatment in response to severity the urinary incontinence.

22. The method as defined in claim 1, further comprising administering a beta-blocker that is responsive to reduce tachycardia, a skeletal muscle relaxant, a narcotic, a proton pump inhibitor, or two or more thereof.

23. The method as defined in claim 1, wherein the tricyclic antidepressant is administered in an amount in a range of about 0.1 mg/kg/day to about 2.5 mg/kg/day.

24. The method as defined in claim 23, wherein the tricyclic antidepressant is administered in an amount in a range of about 0.5 mg/kg/day to about 2 mg/kg/day.

25. The method as defined in claim 1, wherein the tricyclic antidepressant to stool softener ratio is in a range of from about 1:80 to about 3:1.

26. The method as defined in claim 25, wherein the tricyclic antidepressant to stool softener ratio is in a range of from about 1:4 to about 1:3.

27. The method as defined in claim 1, wherein the stool softener is administered in an amount in a range of about 1 mg/kg/day to about 4 mg/kg/day.

28. The method as defined in claim 27, wherein the stool softener is administered in an amount in a range of about 2 mg/kg/day to about 3 mg/kg/day.

29. A medicinal composition for treating a mammal having a urinary disorder comprising urinary incontinence, urinary urgency, or both urinary incontinence and urinary urgency, the composition comprising:
a tricyclic antidepressant, and
a stool softener.

30. The composition as defined in claim 29, wherein the urinary incontinence comprises stress incontinence, urge incontinence, mixed incontinence, post void incontinence, functional incontinence, nocturnal incontinence, or a combination of two or more thereof.

31. The composition as defined in claim 30, wherein the urinary incontinence comprises stress incontinence or functional incontinence.

32. The composition as defined in claim 29, wherein the urinary incontinence is caused by surgery, child-birth, trauma, malnutrition, diabetes, cancer or damage to the spinal cord.

33. The composition as defined in claim 29, wherein the mammal is a human.

34. The composition as defined in claim 33, wherein the mammal is a cat or a dog.

35. The composition as defined in claim 29, wherein the tricyclic antidepressant is present in an amount in a range from about 5 milligrams per total daily dose to about 200 milligrams per total daily dose.

36. The composition as defined in claim 29, wherein the stool softener is present in an amount in a range from about 100 milligrams per total daily dose to about 400 milligrams per total daily dose.

37. The composition as defined in claim 36, wherein the stool softener is present is present in an amount in a range from about 200 milligrams per total daily dose to about 300 milligrams per total daily dose.

38. The composition as defined in claim 29, wherein the tricyclic antidepressant comprises imipramine hydrochloride, imipramine pamoate, a pharmacologically acceptable salt of imipramine, or combinations of two or more thereof.

39. The composition as defined in claim 29, wherein the mammal is a non-elderly adult human.

40. The composition as defined in claim 29, wherein the human is not clinically depressed.

41. The composition as defined in claim 29, wherein the stool softener comprises an anionic surfactant.

42. The composition as defined in claim 41, wherein the surfactant comprises fecal lubricant.

43. The composition as defined in claim 29, further comprising a beta-blocker that is responsive to reduce tachycardia, a skeletal muscle relaxant, a narcotic, a proton pump inhibitor, or two or more thereof.

44. The composition as defined in claim 29, wherein medicine is configured as fractional dosage amounts, the fractional dosage amounts being operable to effect variations in a total daily dosage amount of the tricyclic antidepressant and of the stool softener over a course of treatment.

45. The composition as defined in claim 29, wherein the tricyclic antidepressant and the stool softener in the medicine are configured for packaging to be adjacent to each other in each dose or admixed with each other in each dose for administration substantially simultaneously with each other; or
the tricyclic antidepressant and the stool softener in the medicine are packaged separate from each other for administration substantially simultaneously, sequentially, or alternating periodically with each other.

46. The composition as defined in claim 29, wherein at least a portion of the medicine is in the form of a pill, capsule, gelcap, an ingestible liquid admixture, transdermal patch, an oral or nasal inhalable powder or mist, an enema or suppository, a coated or chewable tablet, a chewable gum, an intravenous solution, or an intramuscular injectable liquid.

47. The composition as defined in claim 29, wherein the medicine is in the form of a plurality of co-packaged dosages of the medicine, and each dose comprises a portion of a daily dosage amount, wherein
each dose comprises the tricyclic antidepressant and the stool softener, or
a first portion of the plurality of dosages comprises the tricyclic antidepressant and not the stool softener, and a second portion of the plurality of dosages includes the stool softener and not the tricyclic antidepressant, and doses of the first portion and the second portion are administrable to form a total daily dose.

48. The composition as defined in claim 29, wherein the tricyclic antidepressant is administered in an amount in a range of about 0.1 mg/kg/day to about 2.5 mg/kg/day.

49. The composition as defined in claim 48, wherein the tricyclic antidepressant is administered in an amount in a range of about 0.5 mg/kg/day to about 2 mg/kg/day.

50. The composition as defined in claim 29, wherein the tricyclic antidepressant to stool softener ratio is in a range of from about 1:80 to about 3:1.

51. The composition as defined in claim 50, wherein the tricyclic antidepressant to stool softener ratio is in a range of from about 1:4 to about 1:3.

52. The composition as defined in claim 29, wherein the stool softener is administered in an amount in a range of about 1 mg/kg/day to about 4 mg/kg/day.

53. The composition as defined in claim 52, wherein the stool softener is administered in an amount in a range of about 2 mg/kg/day to about 3 mg/kg/day.

54. A treatment kit for a mammal having a urinary disorder comprising urinary incontinence, urinary urgency, or both, the kit comprising:
a plurality of doses of a medicine, the medicine comprising:
a tricyclic antidepressant, and
a stool softener comprising one or more of a surfactant and a fecal lubricant.

55. The kit as defined in claim 54, further comprising an instruction set comprising directions for administering the medicine, the instruction set comprising dosage amounts and dosing schedules.

56. A process for treating a gastrointestinal disorder comprising urinary incontinence, urinary urgency, or both in a mammal, the process comprising:
causing an interaction with muscarinic receptors in the mammal to reduce or eliminate at least one symptom caused by or associated with urinary incontinence and/or urinary urgency and affecting a stool of the mammal, and
delivering into fecal matter an oil and water by emulsification using a surfactant to soften the stool of the mammal,
delivering into fecal matter a fecal lubricant to facilitate passage of the stool, or
emulsifying and lubricating the fecal matter to both soften the stool and facilitate passage of the stool,
wherein the emulsifying, lubricating, or emulsifying and lubricating occurs in the bowel of the mammal.

57. The process as defined in claim 56, wherein the step of interacting comprises affecting the bladder inner sphincter.
